(19) United States
(12) Patent Application Publication
(10) Pub. No.: US 2023/0187076 A1
(43) Pub. Date: Jun. 15, 2023

(54) DISEASE RISK PREDICTION METHOD AND SYSTEM BASED ON BIOLOGICAL AGE USING MEDICAL CHECK-UP CLINICAL DATA INDEPENDENT OF DYSLIPIDEMIA DATA

(71) Applicant: MEDIAGE Co.,Ltd, Seongnam-si (KR)

(72) Inventor: Chul Young BAE, Anyang-si (KR)

(21) Appl. No.: 17/752,820

(22) Filed: May 24, 2022

(30) Foreign Application Priority Data
Dec. 3, 2021 (KR) 10-2021-0171965
May 6, 2022 (KR) 10-2022-0056034

(51) Int. Cl. G16H 50/30; G16H 10/60; G16H 50/20

(52) U.S. Cl. CPC G16H 50/30; G16H 10/60; G16H 50/20

(57) ABSTRACT

A system for measuring biological age and predicting a risk of age related disease based on the biological age using biomarkers of general medical check-up regardless of whether or not dyslipidemia test is conducted according to the present disclosure includes an input unit configured to receive, when basic information such as gender/age and biomarker information such as a medical check-up result a customer are provided, the basic information and biomarker information of the customer, a biological age measurement unit configured to measure biological age using the received information, a disease incidence risk prediction unit configured to predict a risk of incidence for age related diseases based on the biological age, an analysis result generation unit configured to generate result report information, a content generation unit, and a service server configured to provide the result report information to a customer.

### CROSS-REFERENCE TO RELATED APPLICATION

This application claims priority to Korean Patent Application No.10-2021- 0171965 filed on Dec. 3, 2021, Korean Patent Application No.10-2022- 0056034 filed on May 6, 2022 and all the benefits accruing therefrom under 35 U.S.C. §119, the contents of which are incorporated by reference in their entirety.

### BACKGROUND

The present disclosure relates to a system for measuring biological age using biomarkers of general medical check-up regardless of whether or not dyslipidemia test is conducted and predicting a risk of incidence for age related disease based on the biological age.

An increase in the elderly population due to rising life expectancy and/or declining birthrates is a global trend. According to the United Nations, if a share of the population aged 65 or older in the whole population is 7% or more, it is classified as an aging society, if the share is 14% or more, it is an aged society, and if the share of the population aged 65 or older is 20% or more, it is classified as a super-aged society. The United States already became an aging society in 1941 and Japan in 1970, and Korea entered an aging society in 2000. The proportion of the elderly population continues to increase, and it is expected that Korea will enter a super-aged society with 20.3% of the share of the population aged 65 or older in 2025.

Age related disease (ARD) is the most common disease that occurs as the frequency increases as aging increases. Examples of age related diseases include cardiovascular disease, arthritis, cataract, osteoporosis, diabetes, high blood pressure, and Alzheimer’s disease. The incidence of all these diseases increases exponentially with age.

Accordingly, there is an urgent need to develop a technology that helps to people to prepare for a healthy life by recognizing the risk of age related disease incidence and predicting the risk of age related disease incidence.

In relation to biological age, the following PTL 1 (Korea Patent No. 10-1328643) provides an apparatus and method for predicting biological age, but a method for predicting disease risk using the apparatus and method for predicting biological age is not known.

Furthermore, conventionally, in the general medical check-up, dyslipidemia-related test items (total cholesterol, triglycerides, high-density lipoprotein cholesterol, low-density lipoprotein cholesterol) were tested once every 4 years for men aged 24 years or older and women aged 40 years or older. For example, only males aged 24, 28, and 32 ..., and females aged 40, 44, and 48 ... correspond to dyslipidemia-related test subjects. For this reason, measuring the biological age and calculating the disease risk in consideration of dyslipidemia causes a problem that has no choice but to use data every 4 years.

### SUMMARY

The present disclosure provides a method and system for measuring biological age, which is actual age of the body compared to resident registration age (age), using biomarkers of general medical check-up regardless of whether the dyslipidemia-related test is conducted or not, and predicting the risk of age related disease incidence based on the measurement of biological age.

The present disclosure also provides a method and system for measuring biological age using biomarkers and providing a customized analysis result corresponding to the measured biological age to a customer. The present disclosure provides a method and system for providing an effect that can help the prevention and management of disease by enabling the customer to easily understand the biological age through the measurement of biological age, as well as providing the risk of age related disease incidence to the customer.

In accordance with an exemplary embodiment, a system for calculating a risk of age related disease based on biological age which is applied to a system for predicting a risk of disease based on biological age, including an input unit configured to receive basic information such as gender and age, and biomarker information including a medical check-up result of a subject; and an analysis unit that comprises a biological age measurement unit configured to calculate biological age of the subject based on the basic information and the biomarker information of the subject, and a disease incidence risk prediction unit configured to predict a risk of incidence for individual diseases based on the biological age of the subject.

In the system, the biomarker information may include at least one or more of height (HT), waist circumference (WC), systolic blood pressure (SBP), fasting blood sugar (FBS), total cholesterol (TC), triglyceride (TG), high-density lipoprotein cholesterol (HDL-C), hemoglobin (Hgb), creatinine (Cr), liver enzyme AST (GOT), liver enzyme γ-GTP (gamma GTP), and estimated glomerular filtration rate (e-GFR), the biological age measurement unit may be configured to determine whether the subject is a male or a female, and whether a dyslipidemia test has been conducted on the subject, calculate basal biological age according to Calculation formula 1 below when the subject is a male and the dyslipidemia test has been conducted on the subject, and calculate the basal biological age according to Calculation formula 2 below when the subject is a male and the dyslipidemia test has not been conducted on the subject, calculate the basal biological age according to Calculation formula 3 below when the subject is a female and the dyslipidemia test has been conducted on the subject, and calculate the basal biological age according to Calculation formula 4 below when the subject is a female and the dyslipidemia test has not been conducted on the subject, and the biological age may be the basal biological age calculated by Calculation formulas 1 to 4 below,

$$\begin{matrix}\text{Biological age = A1 + B1*HT + B2*WC + B3*SBP + B4*FBS +} \\ \text{B5*Hgb + B6*eGFR + B7*AST+ B8*TC + B9*TG +} \\ \text{B10*HDL-C + B11*AGE} \text{(nominal age)}\end{matrix}$$

(A1 is a constant, and B1 to B11 are correlation coefficient values, in which B2, B3, B4, B5, B7, B8, B9, and B11 have positive values and A1, B1, B6, and B10 have negative values),

$$\begin{matrix}\text{Biological age = A2 + B12*HT + B13*WC +} \\ \text{B14*SBP + B15*FBS+ B16*Hgb + B17*eGFR} \\ \text{+ B18*AST+ B19*AGE} \left(\text{nominal age}\right)\end{matrix}$$

(A2 is a constant, B12 to B19 are correlation coefficient values, in which B13, B14, B15, B16, B18, B19 have positive values and A2, B12, and B17 have negative values),

$$\begin{matrix}\text{Biological age = a1 + b1*HT + b2*Wc + b3*SBP + b4*FBS +} \\ \text{b5*TC + b6*TG + b7*HDL-C+ b8*eGFR + b9*AST +} \\ \text{b10*} γ \text{-GTP + b11*AGE (nominal age)}\end{matrix}$$

(a1 is a constant, b1 to b11 are correlation coefficient values, in which b2, b3, b4, b5, b6, b9, b10, b11 have positive values and a1, b1, b7, and b8 have negative values),

$$\begin{matrix}\text{Biological age = a2 + b12*HT + b13*WC +b14*SBP + b15*FBS} \\ \text{+b16*eGFR + b17*AST + b18*} γ \text{-GTP + b19*AGE} \left(\text{nominal age}\right)\end{matrix}$$

(a2 is a constant, B12 to B19 are correlation coefficient values, in which b13, b14, b15, b17, b18, and b19 have positive values and a2, b12, and b16 have negative values).

The biological age measurement unit and biological age measurement procedure may further include questionnaire information of the subject to calculate a corrected biological age for correcting the calculated basal biological age, wherein the questionnaire information may include information about family history, smoking, drinking, and exercise, and the corrected biological age may be calculated by Calculation formula 5 below

$$\begin{matrix}\text{Corrected biological age = basal biological age +} \left(\text{d + d1*family}\right) \\ \left(\text{history + d2*smoking + d3*drinking + d4*exercise}\right)\end{matrix}$$

(the family history is information about presence or absence of a family history, smoking is information about YES or NO status about smoking and pack year, drinking is information about YES or NO status about drinking and an amount of alcohol drinking per day, exercise is information about an amount of exercise per week, d is a constant obtained through regression analysis between a difference between the biological age and the nominal age and family history, smoking, drinking, and exercise information, and d1 to d4 are correlation coefficient values obtained by performing regression analysis on a correlation between the difference between the biological age before correction and the nominal age, and family history, smoking, drinking, and exercise information).

The disease incidence risk prediction procedure and disease incidence risk prediction unit may calculate a risk of individual disease of at least one or more of risks of dementia, prostate disease, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes incidence when the subject is a male, and calculate the risk of individual disease of at least one or more of risks of dementia, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes incidence when the subject is a female, wherein the risk of individual disease is calculated by multiplying the biological age of the subject by a value of relative risk of individual disease.

The disease incidence risk prediction unit and prediction procedure predict a risk of incidence for individual diseases based on the corrected biological age.

The present invention also includes a computer server for performing a method for calculating a risk of disease incidence based on biological age and a service server for transmitting a calculated risk of disease incidence based on biological age via a communication network, and provides a recording medium loaded with the computer program.

The present disclosure provides an effect of enabling the customer to easily understand the health condition of his/her body by measuring the biological age, which is actual age of the body compared to resident registration age using biomarkers of general medical check-up regardless of whether the dyslipidemia test is conducted, and provides an effect that can help the customer to prevent disease incidence or maintain and manage lifestyles by providing information obtained by predicting the risk of age related disease incidence according to the increase in biological age based on the measured biological age.

This can be used for the application of a health care field in the general local community or for providing a customized management service to the customer, and can be used for the provision of a product and content through WEB or APP of a method of predicting the risk of disease incidence, thereby capable of further maximizing the effect.

### BRIEF DESCRIPTION OF THE DRAWINGS

FIG. 1 is a schematic configuration diagram of a system for measuring biological age using biomarkers of general medical check-up regardless of whether or not dyslipidemia test is conducted, and predicting a risk of age related disease incidence based on the biological age.

FIG. 2 is a schematic flowchart of the system for measuring biological age using biomarkers of general medical check-up regardless of whether or not dyslipidemia test is conducted, and predicting the risk of age related disease incidence based on the biological age.

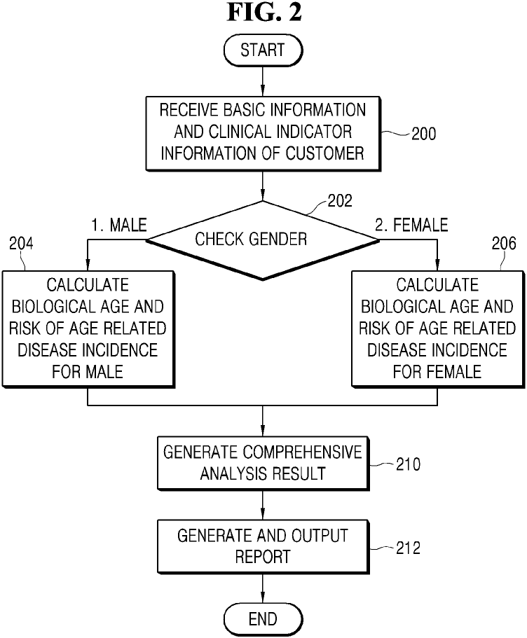

### DETAILED DESCRIPTION OF EMBODIMENTS

A method for measuring biological age and a method for predicting a risk of age related disease incidence based on biological age that are applicable to the system for measuring biological age using biomarkers of general medical check-up regardless of whether or not dyslipidemia test is conducted, and predicting a risk of age related disease based on the biological age will be described in detail.

In the present disclosure, age, actual age, nominal age, resident registration age, and legal age are expressions used with substantially the same meaning, and are the age calculated based on the time at which a person is born. In contrast, bio-age/biological age, unlike the actual age and the nominal age described above, is the age calculated according to an age calculation method of the present disclosure, and the biological age may be calculated differently even for a person of the same actual age depending on a health status and an aging status.

### 1. Method and System for Measuring Biological Age According to The Present Disclosure

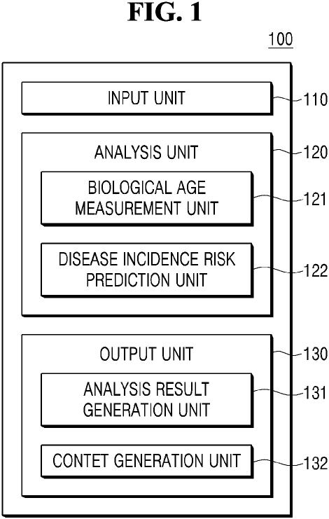

The system of the present disclosure illustrated in a block diagram in FIG. 1 receives basic information of a target customer and biomarker information for the customer through an input unit 110 for predicting the risk of age related disease incidence based on the biological age for the customer. The input device of the user may include a device of a user (customer), an input page received from an APP within the device, or a WEB or API server of a service provider, and may be a terminal device of a service provider system.

The basic information of the customer includes a name, gender, and age of the customer.

The biomarker information includes height (HT), waist circumference (WC), systolic blood pressure (SBP), fasting blood sugar (FBS), total cholesterol (TC), triglyceride (TG), high-density lipoprotein cholesterol (HDL-C), hemoglobin (Hgb), creatinine (Cr), liver enzyme AST (GOT), liver enzyme γ-GTP (gamma GTP), and estimated glomerular filtration rate (e-GFR), etc.

When basic information of the customer and biomarker information for the customer are provided (step 200), it is checked whether gender information included in the basic information of the customer indicates a male or a female (step 202). When the customer indicates a male, a biological age measurement unit 121 of an analysis unit 120 measures the biological age of the customer through biomarker information about the customer according to Calculation formulas 1 and 2 (step 204). Here, the biomarker information includes height (HT), waist circumference (WC), systolic blood pressure (SBP), fasting blood sugar (FBS), hemoglobin (Hgb), liver enzyme AST (GOT), estimated glomerular filtration rate (e-GFR), total cholesterol (TC), triglyceride (TG), high-density lipoprotein cholesterol (HDL-C). Calculation formulas 1 and 2 are for calculating the biological age, and when the dyslipidemia test is conducted, Calculation formula 1 is used, and when the dyslipidemia test is not conducted, Calculation formula 2 is used. In order to calculate the biological age divided as described above, there may be a procedure for checking dyslipidemia test information.

$$\begin{matrix}\text{Biological age = A1 + B1*HT + B2*WC + B3*SBP + B4*FBS +B5*} \\ \text{Hgb + B6*eGFR + B7*AST+ B8*TC + B9*TG +} \\ \text{B10*HDL-C + B11*AGE (nominal age)}\end{matrix}$$

$$\begin{matrix}\text{Biological age = A2 + B12*HT + B13*WC +B14*SBP + B15*FBS+} \\ \text{B16*Hgb + B17*eGFR + B18*AST+ B19*AGE} \left(\text{nominal age}\right)\end{matrix}$$

Calculation formulas 1 and 2 above are obtained by multiplying a different correlation coefficient for each of the biomarker information and then adding the multiplication results.

In Calculation formula 1 above, A1 is a constant, and B1 to B11 are preset values, are correlation coefficient values obtained by statistically analyzing biomarker information (sample data) for a plurality of men prepared in advance for the present disclosure, and may have negative values or positive values according to biomarker information. That is, in Calculation formula 1 above, B2, B3, B4, B5, B7, B8, B9, and B11 may have positive values and A1, B1, B6, and B10 may have negative values.

In Calculation formula 2 above, A2 is a constant, and B12 to B19 are preset values, are correlation coefficient values obtained by statistically analyzing biomarker information (sample data) for a plurality of men prepared in advance for the present disclosure, and have negative values or positive values according to biomarker information. That is, in Calculation formula 2 above, B13, B14, B15, B16, B18, and B19 may have positive values and A2, B12, and B17 may have negative values.

The correlation coefficients are correlation coefficients between nominal age and clinical parameters, may be obtained from sample clinical data through multiple regression analysis for sample subjects whose nominal age and biomarker information are known. For a male, the indicators most highly correlated with age were height (HT), hemoglobin (Hgb), and waist circumference (WC) (r = -0.478 and -0.321 and -0.276; p < 0.0001).

Contrary to the above, when the gender information included in the basic information of the customer indicates a female, the biological age measurement unit 121 of the analysis unit 120 measures basal biological age of the customer through the biomarker information for the customer according to Calculation formulas 3 and 4 (step 206). Here, the biomarker information includes height (HT), waist circumference (WC), systolic blood pressure (SBP), fasting blood sugar (FBS), total cholesterol (TC), triglyceride (TG), high-density lipoprotein cholesterol (HDL-C), estimated glomerular filtration rate (e-GFR), liver enzyme AST (GOT), and liver enzyme γ-GTP (gamma GTP). Calculation formulas 1 and 2 are for calculating the biological age, and when the dyslipidemia test is conducted, Calculation formula 3 is used, and when the dyslipidemia test is not conducted, Calculation formula 4 is used. In order to calculate the biological age divided as described above, there may be a procedure for checking dyslipidemia test information.

$$\begin{matrix}\text{Biological age = a1 + b1*HT + b2*WC + b3*SBP + b4*FBS +b5*} \\ \text{TC + b6*TG + b7*HDL-C+ b8*eGFR + b9*AST + b10*} γ \text{-GTP +} \\ \text{b11*AGE (nominal age)}\end{matrix}$$

$$\begin{matrix}\text{Biological age = a2 + b12*HT + b13*WC +b14*SBP + b15*FBS +} \\ \text{b16*eGFR + b17*AST + b18*} γ \text{-GTP + b19*AGE} \left(\text{nominal age}\right)\end{matrix}$$

Calculation formulas 3 and 4 above are obtained by multiplying a different correlation coefficient for each of the biomarker information and then adding the multiplication results.

In Calculation formula 3 above, a1 is a constant, and b1 to b11 are preset values, are correlation coefficient values obtained by statistically analyzing biomarker information for a plurality of women prepared in advance for the present disclosure, and have negative values or positive values according to biomarker information. That is, in Calculation formula 3 above, b2, b3, b4, b5, b6, b9, b10, and b11 have positive values and a1, b1, b7, and b8 have negative values.

In Calculation formula 4 above, a2 is a constant, and b12 to b19 are preset values, are correlation coefficient values obtained by statistically analyzing biomarker information for a plurality of women prepared in advance for the present disclosure, and have negative values or positive values according to biomarker information. That is, in Calculation formula 4 above, b13, b14, b15, b17, b18, and b19 have positive values and a2, b12, and b16 have negative values.

The correlation coefficients are correlation coefficients between nominal age and clinical parameters, may be obtained from sample clinical data through multiple regression analysis for sample subjects whose nominal age (AGE) and biomarker information are known. For a female, the indicators most highly correlated with age were height (HT), and waist circumference (WC), and systolic blood pressure (SBP) (r = -0.549 and 0.435 and 0.391; p < 0.0001).

According to the correlation coefficient obtained as above, for example, the waist circumference (WC) of the customer can be interpreted as increasing the biological age by a value multiplied by B2 or B13 for a male and increasing the biological age by a value multiplied by b2 or b13 for a female, and the height (HT) of the customer can be interpreted as decreasing the biological age by a value multiplied by B1 or B12 for a male and decreasing the biological age by a value multiplied by b1or b12 for a female.

Meanwhile, the input unit may additionally receive questionnaire information of the subject including information on family history, smoking, drinking, and exercise, the biological age measurement unit may calculate a corrected biological age for correcting the calculated biological age, and the corrected biological age can be calculated by Calculation formula 5 below.

In the correction of biological age, when the calculated biological age is basal biological age, the corrected biological age is calculated by using YES or NO status about smoking, pack year, YES or NO status about drinking, amount of alcohol drinking per day, amount of exercise per week, and family history, which are questionnaire information, as variables for the calculated basal biological age (step 206).

$$\begin{matrix}\text{Corrected biological age = basal biological age +} \left(\text{d + d1*family}\right) \\ \left(\text{history + d2*smoking + d3*drinking + d4*exercise}\right)\end{matrix}$$

d is a constant obtained through regression analysis between a difference between the biological age and the nominal age and family history, smoking, drinking, and exercise information, and d1 to d4 are preset correlation coefficient values, family history refers to information about the presence or absence of family history of the disease, smoking refers to information about YES or NO status about smoking and pack year, drinking refers to information about YES or NO status about drinking and amount of alcohol drinking per day, and exercise refers to amount of exercise per week).

The preset values d and d1 to d4 are a constant and correlation coefficient values generated through multiple regression analysis from sample data according to Calculation formula 6 below, respectively. That is, a, and b1 to b4 may be a constant and correlation coefficient values obtained by performing multiple regression analysis on (basal biological age - nominal age), family history, smoking, drinking, and exercise information in the sample data, in which family history, smoking, drinking, exercise information, basal biological age, and nominal age are known, respectively.

$$\begin{matrix}\text{Basal biological age - nominal age = d + d1*family history +} \\ \text{d2*smoking + d3*drinking + d4*exercise}\end{matrix}$$

The YES or NO status about smoking described above is classified according to whether or not the person has smoked more than 5 packs (100 cigarettes) in his/her lifetime, and pack year (amount of cigarettes smoked per day and number of years smoked. pack and year) is calculated as the number of packyears before quitting smoking if the person smoked in the past but does not smoke now, and if the person is still smoking, the pack year is calculated as “pack-year = (pack/day) x years”. The YES or NO status about drinking and amount of alcohol drinking per day are calculated using “Alcohol intake per day g/day = frequency of intake x intake per drink x alcohol content of soju 22% x 0.8 g” according to an alcohol content calculation formula “[Intake (mL) x Alcohol content(%) x 0.8 (Alcohol specific gravity)]/100” to determine how many days a week a person drinks on average and how much a person drinks per day when drinking (regardless of the type of alcohol, 1 can of beer (355 cc) equals 1.6 glasses of beer), and are classified according to the WHO daily alcohol standard. Also, in the case of exercise, the amount of activity for one week may be calculated as “total activity for one week = ∑ each physical activity MET x MIN”, and each physical activity is divided into strenuous physical activity, moderate-intensity physical activity, and walking, where the strenuous physical activity may be calculated as “number of times per week x 8.0 MET x 60 minutes”, the moderate-intensity physical activity as “number of times per week x 4.0 MET x 60 minutes”, and the walking as “number of times per week x 3.3 MET x 30 minutes”.

### 2. Method of Predicting the Risk of Age Related Disease Incidence Based on Biological Age

When basic information of the customer and biomarker information for the customer is provided (step 200), a disease incidence risk prediction unit 122 of the analysis unit 120 of the system of the present disclosure checks whether gender information included in the basic information of the customer (subject) indicates a male or a female (step 202).

When the customer is indicated as a male, the disease incidence risk prediction unit 122 of the analysis unit 120 measures the risk of age related disease incidence of the customer through the biological age measured above according to Calculation formula 7 (step 204). Here, age related diseases may include a total of 16 types of dementia, prostate disease, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes.

$$\text{Risk of individual disease incidence = Ci*biological age}$$

In Calculation formula 7 above, the individual diseases include dementia, prostate disease, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes, and and Ci is a value of relative risk for each individual disease.

Calculation formula 7 can be expressed as follows for each individual disease.

$$\text{Risk of dementia incidence = C1*biological age}$$

$$\text{Risk of prostate disease incidence = C2*biological age}$$

$$\text{Risk of osteoporosis incidence = C3*biological age}$$

$$\text{Risk of chronic obstructive pulmonary disease incidence =C4*biological age}$$

$$\text{Risk of Parkinson’s disease incidence = C5*biological age}$$

$$\text{Risk of cataract incidence = C6*Body age}$$

$$\text{Risk of macular degeneration incidence = C7*biological age}$$

$$\text{Risk of fracture incidence = C8*biological age}$$

$$\text{Risk of osteoarthritis incidence = C9 *biological age}$$

$$\text{Risk of high blood pressure incidence = C10*biological age}$$

$$\text{Risk of myocardial infarction incidence = C11 *biological age}$$

$$\text{Risk of chronic renal failure incidence = C12 *biological age}$$

$$\text{Risk of hyperlipidemia incidence = C13*biological age}$$

$$\text{Risk of obesity incidence = C14 *biological age}$$

$$\text{Risk of stroke incidence = C15*biological age}$$

$$\text{Risk of diabetes incidence = C16*biological age}$$

In Calculation formula 7, the risks of dementia, prostate disease, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes incidence are calculated by multiplying the risk of each age related disease incidence per 1 year of biological age.

In Calculation formula 7, C1 to C16 are preset values, and are values of the relative risk obtained by statistically analyzing the risk of each age related disease incidence per 1 year of biological age for a number of men prepared in advance for the present disclosure, and each of which has a value less than or greater than 1 depending on the biological age. That is, in Calculation formula 7, C1 to C16 all have a value greater than 1.

Contrary to the above, when the gender information included in the basic information of the customer above indicates a female, the disease risk prediction unit 122 of the analysis unit 120 measures the risk of age related disease incidence of the customer through the biological age measured above according to Calculation formula 8 (step 206). Here, age related diseases include a total of 15 types of dementia, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes.

$$\text{Risk of individual disease incidence = Ci*biological age}$$

In Calculation formula 8 above, the individual diseases include dementia, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes, and ci is a value of relative risk for each individual disease.

Calculation formula 8 can be expressed as follows for each individual disease.

$$\text{Risk of dementia incidence = c1*biological age}$$

$$\text{Risk of osteoporosis incidence = c3*biological age}$$

$$\text{Risk of chronic obstructive pulmonary disease incidence =c4*biological age}$$

$$\text{Risk of Parkinson’s disease incidence = c5*biological age}$$

$$\text{Risk of cataract incidence = c6*Body age}$$

$$\text{Risk of macular degeneration incidence = c7*biological age}$$

$$\text{Risk of fracture incidence = c8*biological age}$$

$$\text{Risk of osteoarthritis incidence = c9 *biological age}$$

$$\text{Risk of high blood pressure incidence = c10*biological age}$$

$$\text{Risk of myocardial infarction incidence = c11 *biological age}$$

$$\text{Risk of chronic renal failure incidence = c12 *biological age}$$

$$\text{Risk of hyperlipidemia incidence = c13*biological age}$$

$$\text{Risk of obesity incidence = c14 *biological age}$$

$$\text{Risk of stroke incidence = c15*biological age}$$

$$\text{Risk of diabetes incidence = c16*biological age}$$

In Calculation formula 8, the risks of dementia, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes are calculated by multiplying the risk of each age related disease incidence per 1 year of biological age.

In Calculation formula 8, c1 and c3 to c16 are preset values, and are values of the relative risk obtained by statistically analyzing the risk of each age related disease incidence per 1 year of biological age for a number of women prepared in advance for the present disclosure, and each of which has a value less than or greater than 1 depending on the biological age. That is, in Calculation formula 8, c1 and c3 to c16 all have a value greater than 1.

The biological age in Calculation formulas 7 and 8 may be basal biological age calculated by the previous Calculation formulas 1 to 4, or may be the corrected biological age obtained by correcting the basal biological age by Calculation formulas 5 and 6.

As a statistical modeling method for calculating each coefficient value in Calculation formula 7 and Calculation formula 8, a Cox proportional hazards model built on the assumption that there is a log-linear relationship between a survival function and a variable was used.

According to this, when xᵢ = {xᵢ₁, ... , xᵢₚ] is a variable for item i, the survival function S(t) is expressed as a time t and a variable Xᵢ as shown in Expression ① below.

If this is applied to the present disclosure, according to a comparative risk calculation formula in Expression ② below, the relative risk when biological age increases from k to (k + 1) by one year is obtained as e^{b1}, and when predetermined sample data having individual cancer onset data is substituted into the Cox proportional hazards model, a model parameter b1 is calculated and the comparative risk e^{b1} is calculated.

Cox proportional hazards model:

$$S \left(t \vert {x}_{i}\right) = {S}_{0} \left(t\right) exp \left({b}_{1} {x}_{ε 1} + ⋯ + {b}_{p} {x}_{ε p}\right)$$

Relative risk:

$$\frac{\frac{S \left(t \vert {x}_{1} = k + 1\right)}{{S}_{0} \left(t\right)}}{\frac{S \left(t \vert {x}_{1} = k\right)}{{S}_{0} \left(t\right)}} = \frac{S \left(t \vert {x}_{1} = k + 1\right)}{S \left(t \vert {x}_{1} = k\right)} = \frac{{e}^{{b}_{1} \left(k + 1\right) + {b}_{2} {x}_{2} + ⋯ + {b}_{p} {x}_{p}}}{{e}^{{b}_{1} \left(k\right) + {b}_{2} {x}_{2} + ⋯ + {b}_{p} {x}_{p}}} = {e}^{{b}_{1}}$$

The Cox proportional hazards model is an analysis technique for examining the effects of various risk factors affecting survival on survival (the period from the time of participation in the study to occurrence of the event). In the present disclosure, the risk factor is the biological age, and the occurrence of an event is applied as the incidence of age related disease. Therefore, the Cox proportional hazards model is a function value calculated according to the numerical values of various risk factors, and the comparative risk is a value calculated to see the effect of the risk factor to be checked among risk factor information of the Cox proportional hazards model on survival. For example, when explaining a process of calculating the risk of hyperlipidemia incidence per year increase in biological age in a male group, if an explanatory variable is biological age, the hyperlipidemia incidence over time is given as S(t) = S₀(t)exp(b₁xᵢ₁) from the Cox proportional hazards model, (xᵢ₁ is biological age of i-th sample data), and when a regression coefficient b1 is calculated, the relative risk of hyperlipidemia incidence per year increase in biological age is calculated from Equation ②, from which the relative risk of hyperlipidemia incidence C13 = e^{b1} is obtained.

As an embodiment of the present disclosure, a serial process of analysis as described above was made using R Studio 3.3.3 version, and the interpretation of the results can be described as Example 1) as follows.

Example 1 Risk of hyperlipidemia incidence per 1 year increase in biological age in male group

Summary of coxph model

| Parameter | coef | exp(coef) | se(coef) | z | Pr(>\|z\|) | 95% | Hazardratio |
|---|---|---|---|---|---|---|---|
| | | | | | | confidence limits | |
| | | | | | | Lower | Upper |
| Biological Age | 0.198 | 1.219 | 0.004 | 43.37 | <0.001 | 1.209 | 1.23 |

Example 1 is the result obtained by analyzing the risk of hyperlipidemia incidence per 1 year increase of biological age in the male group using the Cox proportional hazards model. In the table, coef refers to a regression coefficient of an equation estimated as a coefficient, and the risk of incidence is 1.219, which is exp(coef), and is a value obtained by taking the exponential (exponential function) to the value of coef. That is, it is interpreted as a result that the risk of hyperlipidemia incidence increases by 1.219 times per 1 year increase in biological age. The value of P(>|z|) means the probability of significance of the z value and is less than 0.001, so it can be considered a statistically significant result. In addition, when looking at the 95% Hazard ratio confidence limits, it is 1.209 to 1.23, which does not include 1, and thus it can be considered a significant result.

In the present disclosure, the calculation of the regression coefficient may be calculated in a regression analysis process through a Cox regression analysis method according to the conventional Cox proportional hazards model. In the embodiment of the present disclosure, R Studio version 3.3.3 was used, and data obtained from a follow-up survey of approximately 10 million people at Korean National Health Insurance Service Center for approximately 10 years from 2009 was used as sample data. Meanwhile, it will be obvious to those skilled in the art that the technical idea of the present disclosure is not dependent on specific software or the specific sample data for performing the Cox regression analysis.

As described above, when the biological age measurement and risk of age related disease incidence measurement based on biological age are completed, an output unit 130 generates a comprehensive analysis result for prediction of the risk of age related disease incidence based on the biological age including biological age analysis of the customer (step 210). The result of the comprehensive analysis includes actual age and measured biological age of the customer, result of the risk of each age related disease incidence, and statistical information about disease incidence of the same sex and the same age for each age related disease. Here, the result of the comprehensive analysis is divided into five grades, such as good, caution, warning, risk, and high risk, according to the risk of age related disease incidence of the customer, and includes a pre-determined prevention practice guide corresponding to each result.

When the comprehensive analysis result described above is generated, the output unit 130 generates the biological age and report information of the risk of age related disease incidence based on biological age, and outputs the report information in a form that can be provided to the customer (step 212).

Outputting the report information in the form that can be provided to the customer includes displaying the report information on a screen, providing the report information in a printable file format, or providing the report information in a form of an API.

It is obvious to those skilled in the art that the procedure for calculating the risk of disease based on biological age described above can be implemented and carried out through a computer program. The present disclosure includes the computer program and a recording medium onto which the program is loaded, and a computer device and server which are loaded with the program and perform the procedures described above.

The present disclosure also includes a system and service server for calculating the risk of disease incidence for a subject by performing the method and procedure described above and transmitting the risk of disease incidence to a terminal device possessed by the subject through a communication network.

Although the disease risk prediction method and system based on biological age using medical check-up clinical data independent of dyslipidemia data have been described with reference to the specific embodiments, they are not limited thereto. Therefore, it will be readily understood by those skilled in the art that various modifications and changes can be made thereto without departing from the spirit and scope of the present invention defined by the appended claims.

1. A system for calculating a risk of age related disease based on biological age which is applied to a system for predicting a risk of disease incidence based on biological age, comprising:
an input unit configured to receive basic information such as gender and age, and biomarker information including a medical check-up result of a subject; and
an analysis unit that comprises a biological age measurement unit configured to calculate biological age of the subject based on the basic information and the biomarker information of the subject, and a disease incidence risk prediction unit configured to predict a risk of incidence for individual diseases based on the biological age of the subject.

2. The system for claim 1, wherein
the biomarker information includes at least one or more of height (HT), waist circumference (WC), systolic blood pressure (SBP), fasting blood sugar (FBS), total cholesterol (TC), triglyceride (TG), high-density lipoprotein cholesterol (HDL-C), hemoglobin (Hgb), creatinine (Cr), liver enzyme AST (GOT), liver enzyme γ-GTP (gamma GTP), and estimated glomerular filtration rate (e-GFR),
the biological age measurement unit is configured to
determine whether the subject is a male or a female, and whether a dyslipidemia test has been conducted on the subject,
calculate basal biological age according to Calculation formula 1 below when the subject is a male and the dyslipidemia test has been conducted on the subject, and calculate the basal biological age according to Calculation formula 2 below when the subject is a male and the dyslipidemia test has not been conducted on the subject, and
calculate the basal biological age according to Calculation formula 3 below when the subject is a female and the dyslipidemia test has been conducted on the subject, and calculate the basal biological age according to Calculation formula 4 below when the subject is a female and the dyslipidemia test has not been conducted on the subject, and
the biological age is the basal biological age calculated by Calculation formulas 1 to 4 below,
$$\begin{matrix}\text{Biological age = A1 +B1*HT + B2*WC +} \\ \text{B3*SBP +B4*FBS +B5*Hgb + B6*eGFR +} \\ \text{B7*AST+ B8*TC + B9*TG +B10*HDL-C + B11*AGE} \left(\text{nominal age}\right)\end{matrix}$$
(A1 is a constant, and B1 to B11 are correlation coefficient values, in which B2, B3, B4, B5, B7, B8, B9, and B11 have positive values and A1, B1, B6, and B10 have negative values),
$$\begin{matrix}\text{Biological age = A2 + B12*HT + B13*WC +} \\ \text{B14*SBP + B15*FBS + B16*Hgb +} \\ \text{B17*eGFR + B18*AST+ B19*AGE} \left(\text{nominal age}\right)\end{matrix}$$
(A2 is a constant, B12 to B19 are correlation coefficient values, in which B13, B14, B15, B16, B18, B19 have positive values and A2, B12, and B17 have negative values),
$$\begin{matrix}\text{Biological age = a1 + b1*HT + b2*WC + b3*SBP} \text{} + \\ \text{b4*FBS + b5*TC + b6*TG +b7*HDL-C+ b8*eGFR +} \\ \text{b9*AST + b10*} \text{γ} \text{-GTP + b11*AGE} \left(\text{nominal age}\right)\end{matrix}$$
(al is a constant, b1 to b11 are correlation coefficient values, in which b2, b3, b4, b5, b6, b9, b10, b11 have positive values and a1, b1, b7, and b8 have negative values),
$$\begin{matrix}\text{Biological age = a} 2 \text{+ b12*HT + b13*WC +} \\ \text{b14*SBP + b15*FBS + b16*eGFR +} \\ \text{b17*AST + b18*} \text{γ} \text{-GTP + b19*AGE} \left(\text{nominal age}\right)\end{matrix}$$
(a2 is a constant, b12 to b19 are correlation coefficient values, in which b13, b14, b15, b17, b18, and b19 have positive values and a2, b12, and b16 have negative values).

3. The system for claim 2, wherein
the disease incidence risk prediction unit is configured to
calculate a risk of individual disease of at least one or more of risks of dementia, prostate disease, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes incidence when the subject is a male, and
calculate the risk of individual disease of at least one or more of risks of dementia, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes incidence when the subject is a female,
the risk of individual disease is calculated by multiplying the biological age of the subject by a value of relative risk of individual disease, and
the value of relative risk of individual disease is a value of relative risk calculated by statistically analyzing risks of individual disease incidence per 1 year of biological age.

4. The system for claim 3, wherein
the input unit is configured to additionally receive questionnaire information of the subject,
the biological age measurement unit is configured to calculate a corrected biological age for correcting the calculated basal biological age based on the questionnaire information of the subject,
the questionnaire information includes information about family history, smoking, drinking, and exercise,
the corrected biological age is calculated by Calculation formula 5 below, and
the disease incidence risk prediction unit is configured to predict the risk of incidence for individual diseases based on the corrected biological age,
$$\begin{matrix}\text{Corrected biological age = basal biological age +} \\ \left(\text{d + d1*family history + d2*smoking +}\right) \\ \text{d3*drinking + d4*exercise}\end{matrix}$$
(the family history is information about presence or absence of a family history, smoking is information about YES or NO status about smoking and pack year, drinking is information about YES or NO status about drinking and an amount of alcohol drinking per day, exercise is information about an amount of exercise per week, d is a constant obtained through regression analysis between a difference between the biological age and the nominal age and family history, smoking, drinking, and exercise information, and d1 to d4 are correlation coefficient values obtained by performing regression analysis on a correlation between the difference between the biological age before correction and the nominal age, and family history, smoking, drinking, and exercise information).

5. A method of calculating a risk of disease incidence based on biological age, comprising:
receiving basic information such as gender and age, and biomarker information including a medical check-up result of a disease incidence risk calculation subject;
checking gender information of the subject based on the information input in the receiving of basic information;
calculating biological age of the subject based on the biomarker information; and
calculating a risk of individual disease incidence for the subject based on the calculated biological age.

6. The method of claim 5, wherein
the biomarker information includes at least one or more of height (HT), waist circumference (WC), systolic blood pressure (SBP), fasting blood sugar (FBS), total cholesterol (TC), triglyceride (TG), high-density lipoprotein cholesterol (HDL-C), hemoglobin (Hgb), creatinine (Cr), liver enzyme AST (GOT), liver enzyme γ-GTP (gamma GTP), and estimated glomerular filtration rate (e-GFR),
in the calculating of the biological age,
it is determined whether the subject is a male or a female, and whether a dyslipidemia test has been conducted on the subject, and
the basal biological age is calculated according to Calculation formula 1 below when the subject is a male and a dyslipidemia test has been conducted on the subject, and the basal biological age is calculated according to Calculation formula 2 below when the subject is a male and the dyslipidemia test has not been conducted on the subject, and
the basal biological age is calculated according to Calculation formula 3 below when the subject is a female and the dyslipidemia test has been conducted on the subject, and the basal biological age is calculated according to Calculation formula 4 below when the subject is a female and the dyslipidemia test has not been conducted on the subject,
$$\begin{matrix}\text{Biological age = A1 + B1*HT + B2*WC + B3*SBP +} \\ \text{B4*FBS +} \text{} \text{B5*Hgb + B6*eGFR +B7*AST+ B8*TC +} \\ \text{B9*TG + B10*HDL-C + B11*AGE} \left(\text{nominal age}\right)\end{matrix}$$
(A1 is a constant, and B1 to B11 are correlation coefficient values, in which B2, B3, B4, B5, B7, B8, B9, and B11 have positive values and A1, B1, B6, and B10 have negative values),
$$\begin{matrix}\text{Biological age = A2 + B12*HT + B13*WC} \\ \text{+ B14*SBP + B15*FBS + B16*Hgb +} \\ \text{B17*eGFR + B18*AST+ B19* AGE} \left(\text{nominal age}\right)\end{matrix}$$
(A2 is a constant, B12 to B19 are correlation coefficient values, in which B13, B14, B15, B16, B18, B19 have positive values and A2, B12, and B17 have negative values),
$$\begin{matrix}\text{Biological age = a1 + b1*HT + b2*WC +} \\ \text{b3*SBP + b4*FBS + b5*TC + b6*TG +b7*HDL-C+} \\ \text{b8*eGFR + b9*AST + b10*} \text{γ} \text{-GTP + b11*AGE} \left(\text{nominal age}\right)\end{matrix}$$
(al is a constant, b1 to b11 are correlation coefficient values, in which b2, b3, b4, b5, b6, b9, b10, b11 have positive values and a1, b1, b7, and b8 have negative values),
$$\begin{matrix}\text{Biological age = a2 + b12*HT + b13*WC} \\ \text{+ b14*SBP + b15*FBS + b16*eGFR +} \\ \text{b} 17 * \text{AST + b18*} \text{γ} \text{-GTP + b19*AGE} \left(\text{nominal age}\right)\end{matrix}$$
(a2 is a constant, b12 to b19 are correlation coefficient values, in which b13, b14, b15, b17, b18, and b19 have positive values and a2, b12, and b16 have negative values).

7. The method of claim 6, wherein
in the calculating of the risk of individual disease incidence,
a risk of individual disease of at least one or more of risks of dementia, prostate disease, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes incidence is calculated when the subject is a male, and
a risk of individual disease of at least one or more of risks of dementia, osteoporosis, chronic obstructive pulmonary disease, Parkinson’s disease, cataract, macular degeneration, fracture, osteoarthritis, high blood pressure, myocardial infarction, chronic renal failure, hyperlipidemia, obesity, stroke, and diabetes incidence is calculated when the subject is a female,
the risk of individual disease is calculated by multiplying the biological age of the subject by a value of relative risk of individual disease, and
the value of relative risk of individual disease is a value of relative risk calculated by statistically analyzing risks of individual disease incidence per 1 year of biological age.

8. The method of claim 7, wherein
in the receiving of the basic information, questionnaire information of the subject is additionally received,
the calculating of the biological age further comprises calculating a corrected biological age for correcting the calculated biological age based on the questionnaire information of the subject,
the questionnaire information includes information about family history, smoking, drinking, and exercise,
the corrected biological age is calculated by Calculation formula 5 below, and
in the calculating of the risk of individual disease incidence, the risk of incidence for individual diseases is calculated based on the corrected biological age,
$$\begin{matrix}\text{Corrected biological age = basal biological age +} \\ \left(\text{d + d1*family history + d2*smoking +}\right) \text{d3*drinking + d4*exercise}\end{matrix}$$
(the family history is information about presence or absence of a family history, smoking is information about YES or NO status about smoking and pack year, drinking is information about YES or NO status about drinking and an amount of alcohol drinking per day, exercise is information about an amount of exercise per week, d is a constant obtained through regression analysis between a difference between the biological age and the nominal age and family history, smoking, drinking, and exercise information, and d1 to d4 are correlation coefficient values obtained by performing regression analysis on a correlation between the difference between the biological age before correction and the nominal age, and family history, smoking, drinking, and exercise information).

9. A recording medium loaded with a computer program for performing the method of calculating a risk of disease incidence based on biological age according to claims 5.

10. A computer server loaded with a computer program for performing the method of calculating a risk of disease incidence based on biological age according to claims 5.

11. A service server for transmitting a risk of cancer incidence based on biological age calculated through the method of calculating a risk of disease incidence based on biological age according to claims 5 to the subject through a communication network.
